(19) United States
(12) Patent Application Publication
(10) Pub. No.: US 2025/0325752 A1
(43) Pub. Date: Oct. 23, 2025

(54) METHODS AND DEVICES FOR INJECTING HOT MATERIAL INTO SKIN

(71) Applicant: KOLORPEN LTD., Afula (IL)

(72) Inventor: Reuven GAMLIEL, Afula (IL)

(73) Assignee: KOLORPEN LTD., Afula (IL)

(21) Appl. No.: 18/868,179

(22) PCT Filed: May 24, 2023
(86) PCT No.: PCT/IL2023/050536

§ 371 Date: Nov. 22, 2024

Related U.S. Application Data
(60) Provisional application No. 63/345,048, filed on May 24, 2022.

(51) Int. Cl. A61M 5/30; A61M 5/44

(52) U.S. Cl. CPC A61M 5/30; A61M 5/445

(57) ABSTRACT

Embodiments of the invention provided methods and systems for collagen remodelling of an area of skin of a subject using a collagen remodelling device. The method may comprise heating, by a heater unit of the device, a liquid to a temperature configured to cause micro burns to the skin of the subject when the liquid is in contact with the skin of the subject and injecting the liquid, using a needleless injection unit of the device, from a liquid reservoir of the device into the area of skin of the subject.

### FIELD OF THE INVENTION

The present invention relates generally to methods and devices for injecting hot material into skin without the use of needles. Some embodiments of the invention relate to cosmetics. The devices and methods herein may relate to collagen remodelling of an area of skin of a subject.

### BACKGROUND OF THE INVENTION

Signs of human aging may occur in the skin, and may be particularly conspicuous on the face. Two substances, collagen and elastin, may provide skin with elasticity and may prevent wrinkles from forming. As humans age, their collagen production slows down, and as a result their skin may lose its elasticity, leading to deformations and creation of wrinkles. Skin anti-aging treatments often focus on the rehabilitation of collagen fibres, a process called ‘collagen remodelling’.

Collagen remodelling may be stimulated by injuries or microinjuries. Treatments usually attempt to minimise injury trauma, while maximising collagen remodelling. It may be desirable to control the injury strength and control the region in the skin in which the injuries occur.

Various treatments may use various means to cause injuries by burns, such as micro-burns. Such burns may be caused by depositing energy in the skin by means of laser radiation, radio-frequency radiation, or ultrasound. With these means it may be both difficult to control the exact amount of energy deposited and to localise it to the required depth. Such treatments can often damage the skin surface, causing visible wounds and discomfort.

### SUMMARY OF THE INVENTION

Embodiments may improve collagen remodelling technology by using needleless injection of hot material in the skin. Embodiments disclosed herein may allow for improved control over the amount of energy deposited and the localisation of the energy.

Aspects of the invention provide methods for collagen remodelling of an area of skin of a subject using a collagen remodelling device, which may comprise: heating, by a heater unit of the device, a liquid to a temperature configured to cause micro-burns to the skin of the subject when the liquid is in contact with the skin of the subject; and injecting the liquid, using a needleless injection unit of the device, from a liquid reservoir of the device into the area of skin of the subject.

In some embodiments, the needleless injection unit comprises a handheld needleless injection pen configured to be held by a user.

In some embodiments, heating, by a heater unit of the device, a liquid to a temperature configured to cause micro-burns to the skin of the subject when the liquid is in contact with the skin of the subject comprises: positioning the needleless injection unit in an opening of the heater unit; passing an alternating current through at least one conductive coil, by a connected alternating current source, wherein the at least one conductive coil surrounds the opening, and thereby creating an alternating magnetic field in the opening; heating, by the alternating magnetic field, a conductive or semi-conductive element of the needleless injection unit; and transferring heat, from the conductive element, to the liquid stored in the liquid reservoir, wherein the liquid reservoir is housed inside the needleless injection unit.

In some embodiments, the liquid comprises a therapeutic serum.

In some embodiments, the methods further comprise: activating, by a switch unit of the device for operation by a user, the needleless injection unit to allow injection of liquid; and deactivating, by the switch unit, the needleless injection unit to disallow injection of liquid.

In some embodiments, the switch unit comprises a foot pedal.

In some embodiments, the temperature configured to cause micro-burns to the skin of the subject is between 65° C.-80° C.

Aspects of the invention provide collagen remodelling devices for remodelling collagen in an area of skin of a subject, which may comprise: a heater unit, for heating a liquid to a temperature configured to cause micro-burns to the skin of the subject when the liquid is in contact with the skin of the subject; a liquid reservoir; and a needleless injection unit, for injecting the liquid from the liquid reservoir into the area of skin of the subject.

In some embodiments, the needleless injection unit comprises a handheld needleless injection pen configured to be held by a user.

In some embodiments, the liquid reservoir is housed inside the needleless injection unit; the needleless injection unit comprises a conductive or semi-conductive element configured to heat under an alternating magnetic field and transfer heat to the liquid stored in the liquid reservoir; and the heater unit comprises: an opening configured to house the needleless injection unit during heating of the liquid; a temperature sensor; and at least one conductive coil surrounding the opening and connected to an alternating current source, wherein the at least one conductive coil is configured to create an alternating magnetic field in the opening when an alternating current passes through it.

In some embodiments, the liquid comprises a therapeutic serum.

In some embodiments, the device may further comprise a switch unit for operation by a user, wherein the switch unit has an active state configured to cause the needleless injection unit to inject liquid, and an inactive state configured to cause the needleless injection unit to not inject liquid.

In some embodiments, the switch unit comprises a foot pedal.

In some embodiments, the temperature configured to cause micro-burns to the skin of the subject is between 65° C.-80° C.

In some embodiments, the device may further comprise a control unit comprising a processor and a memory.

Aspects of the invention provide methods for improving the bodily appearance of an animal by remodelling collagen of an area of skin of the animal using a collagen remodelling device, which may comprise: heating, by a heater unit of the device, a liquid to a temperature configured to cause micro-burns to the skin of the animal when the liquid is in contact with the skin of the animal; and injecting the liquid, using a needleless injection unit of the device, from a liquid reservoir of the device into the area of skin of the animal.

In some embodiments, the animal is a human.

In some embodiments, the needleless injection unit comprises a handheld needleless injection pen configured to be held by a user.

In some embodiments, heating, by a heater unit of the device, a liquid to a temperature configured to cause micro-burns to the skin of the animal when the liquid is in contact with the skin of the animal comprises: positioning the needleless injection unit in an opening of the heater unit; passing an alternating current through at least one conductive coil, by a connected alternating current source, wherein the at least one conductive coil surrounds the opening, and thereby creating an alternating magnetic field in the opening; heating, by the alternating magnetic field, a conductive or semi-conductive element of the needleless injection unit; and transferring heat, from the conductive element, to the liquid stored in the liquid reservoir, wherein the liquid reservoir is housed inside the needleless injection unit.

In some embodiments, the liquid comprises a serum for reducing the appearance of micro-burns.

In some embodiments, the methods further comprise activating, by a switch unit of the device for operation by a user, the needleless injection unit to allow injection of liquid; and deactivating, by the switch unit, the needleless injection unit to disallow injection of liquid.

In some embodiments, the switch unit comprises a foot pedal.

In some embodiments, the temperature configured to cause micro-burns to the skin of the animal is between 65° C.-80° C.

### BRIEF DESCRIPTION OF THE DRAWINGS

The subject matter regarded as the invention is particularly pointed out and distinctly claimed in the concluding portion of the specification. The invention, however, both as to organization and methods of operation, together with objects, features, and advantages thereof, may best be understood by reference to the following detailed description when read with the accompanying drawings in which:

FIG. 1 shows a flowchart of a method according to embodiments of the present invention.

FIG. 2 shows a schematic diagram of a collagen remodelling device according to embodiments of the invention.

FIG. 3 shows a schematic diagram of a collagen remodelling device controller according to embodiments of the invention.

FIG. 4 shows a schematic diagram of a needleless injection unit of a collagen remodelling device according to embodiments of the invention.

FIG. 5 shows a schematic diagram of a heater unit of a collagen remodelling device according to embodiments of the invention.

FIG. 6 shows a schematic diagram of a collagen remodelling device controller according to embodiments of the invention.

FIG. 7 shows a block diagram of an exemplary computing device which may be used with embodiments of the present invention.

### DETAILED DESCRIPTION OF THE INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analysing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

As used herein, “serum” may refer to a water-based or oil-based formulation, which may contain additional ingredients which may be active ingredients. Serums may be cosmetic products (cosmetic serums) which may in some embodiments perform cosmetic functions in order to improve the bodily appearance of a subject. For example, a serum may act to reduce dullness, fine lines, hyperpigmentation, blemishes, wrinkles, or sagging or aging of the skin. Additionally or alternatively, in some embodiments, serums may perform a therapeutic or medical function. For example, a serum may act to treat acne, rosacea, or eczema. A function performed by a serum may be defined by its ingredients, and their dosages or concentrations. Common ingredients of serums may include: hyaluronic acid, glycolic acid, vitamin C, retinol, niacinamide, peptides, ceramides, alpha-hydroxy acids (AHAs), beta-hydroxy acids (BHAs), plant extracts, and antioxidants.

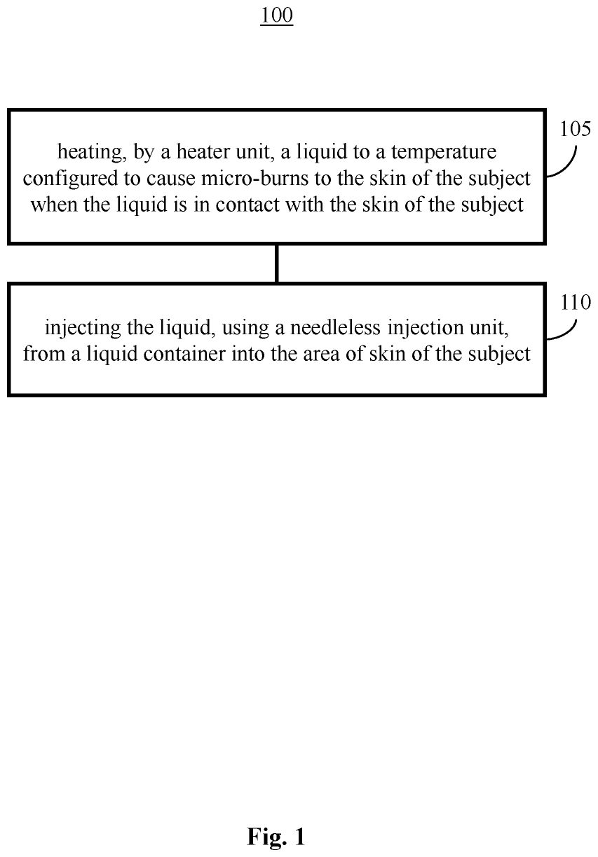

FIG. 1 shows a flowchart of a method 100 for a method of collagen remodelling of an area of skin of a subject using a collagen remodelling device. Method 100 may be suitable for improving the bodily appearance of the subject, for example reducing the appearance of dullness, fine lines, hyperpigmentation, blemishes, wrinkles, or sagging or aging of the skin. For example, method 100 may be suitable for improving the bodily appearance of a face of the subject. In some embodiments, the subject may be an animal, a mammal, and/or a human.

In operation 105, a liquid or fluid may be heated by a heater unit to a temperature configured to cause micro-burns to the skin of the subject when the liquid is in contact with the skin of the subject.

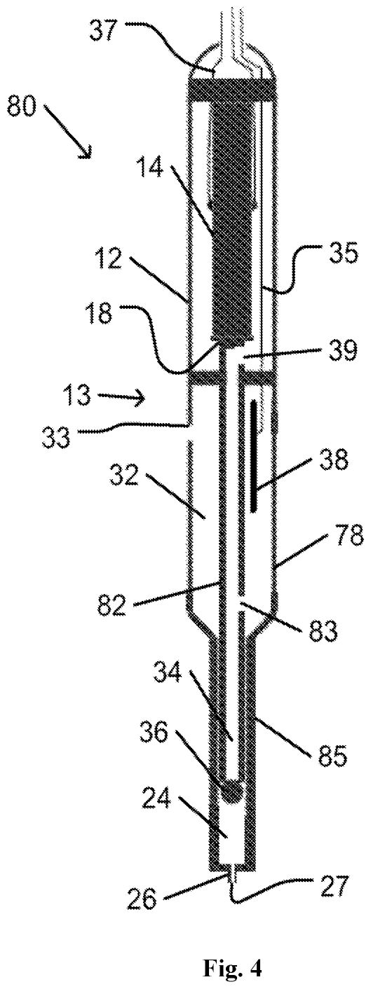

In some embodiments, operation 105 may comprise a number of separate steps. For example, in embodiments with a compact needleless injection unit construction (e.g., as shown in FIGS. 4-6), operation 105 may include any or all of steps (a)-(d).

Step (a) may include positioning the needleless injection unit in an opening of the heater unit. The opening may be configured to house, contain, and/or hold the needleless injection unit.

Step (b) may include passing an alternating current through at least one conductive coil, by a connected alternating current source, wherein the at least one conductive coil surrounds the opening, and thereby creating an alternating magnetic field in the opening.

Step (c) may include heating, by the alternating magnetic field, a conductive or semi-conductive element of the needleless injection unit. The alternating magnetic field may induce eddy currents or hysteresis losses in the conductive or semi-conductive element of the needleless injection unit to heat the element.

Step (d) may include transferring heat, from the conductive element, to the liquid stored in a liquid reservoir or container, wherein the liquid reservoir is housed inside the needleless injection unit.

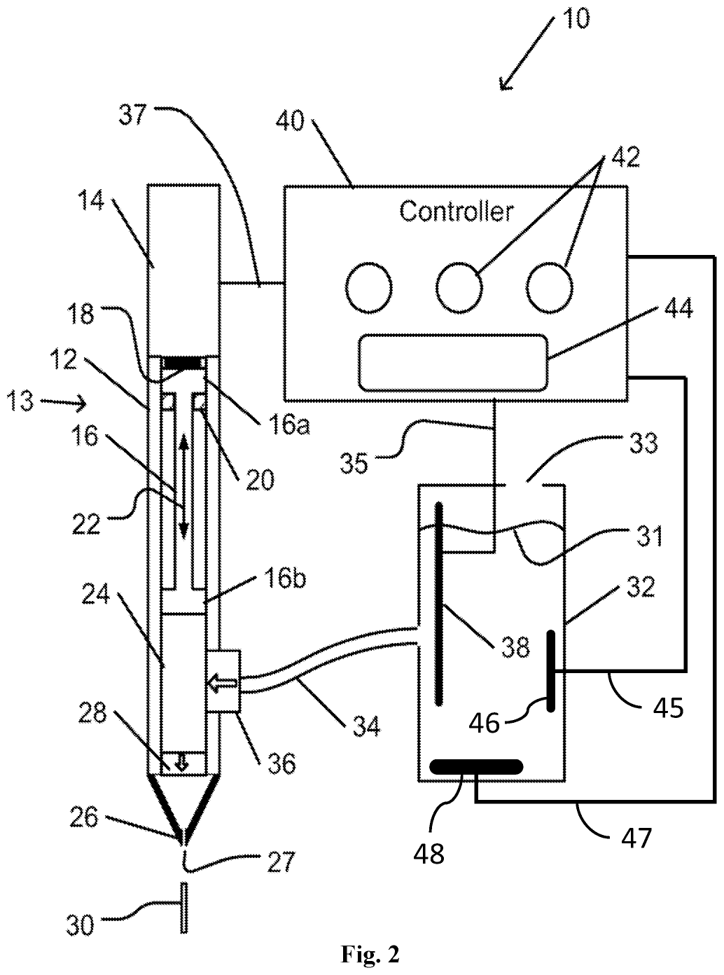

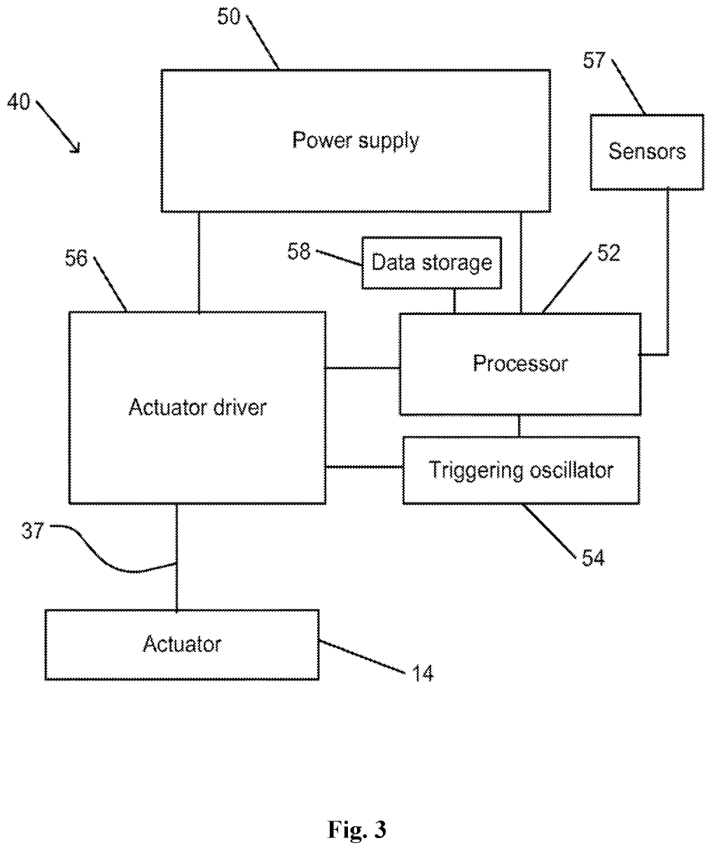

In embodiments with a separated needleless injection unit construction (e.g., as shown in FIGS. 2-3), operation 105 may, for example, include passing current through a heating filament situated in a reservoir holding the liquid, which may transfer heat to the liquid.

The temperature to which the liquid is heated may be controllable or selectable (e.g., by a controller). The temperature may be increased to increase the strength of the injury or micro-burn that the injection step produces. This may, in some cases, increase production of collagen in the skin. It may additionally cause greater injury trauma. Conversely, the temperature may be decreased to decrease the strength of the injury or micro-burn that the injection step produces. This may, in some cases, cause less injury trauma. It may additionally decrease production of collagen in the skin. For a given subject and/or procedure, there may exist an optimal temperature for the liquid. In some cases, the optimal temperature may depend on characteristics of the subject, e.g., the subject's age, properties of the subject's skin, etc. and/or on characteristics of the procedure, e.g., what level of collagen remodelling is required or desired. Present methods of collagen remodelling using needleless injection of hot liquid, wherein the temperature of the liquid may be controlled, may allow for substantially improved control over the amount of energy deposited in the skin when compared to alternative methods. The optimal temperature may lie between 65° C.-80° C.

In operation 110, the liquid may be injected, using a needleless injection unit, from a liquid reservoir into the area of skin of the subject. Operation 110 may additionally or alternatively be stated as administering the liquid using a needleless injection unit from a liquid reservoir into the area of skin of the subject. The needleless injection unit may be configured to inject or administer liquid by directing pressurised micro-jets of the liquid into the skin. The micro-jets may be administered at a specific frequency, e.g., as decided by an impulse generator or actuator. The needleless injection unit may be configured to inject or administer the micro-jets at a specific speed and/or pressure.

In some embodiments, injecting hot liquid into an area of skin of the subject may cause burns in the skin of the subject. The burns may be micro-burns. The burns may be or cause damage to the skin. The liquid may be injected with a velocity that is sufficient to allow the liquid to penetrate to a depth below the surface of the skin. As such, the damage to the skin may be below the surface of the skin. The body of the subject may react to the damage by encouraging or stimulating collagen production or growth in the affected area.

During injection of the hot liquid, a user may move or maintain the needleless injection unit on or above the skin of the subject. The user may activate the injection using a switch unit, button, or similar. For example, the user may, while the needleless injection unit is injecting hot liquid, move the needleless injection unit over an area of skin of the subject which it is intended to improve the appearance of. For example, the needleless injection unit may be used to inject hot liquid in an area of a subject's face which contains wrinkles, in order to stimulate collagen remodelling in this area of the subject's skin.

The speed and/or pressure of the micro-jets of liquid may be controllable or selectable (e.g., by a controller). The speed and/or pressure of the micro-jets may be increased to increase the depth into the skin to which the liquid micro-jet reaches, thus also increasing the depth of the damage caused by the liquid. Conversely, the speed and/or pressure may be decreased to decrease the depth into the skin to which the liquid micro-jet reaches, thus also decreasing the depth of the damage caused by the liquid. For a given subject and/or procedure, there may exist an optimal depth of damage or micro-burns. The speed and/or pressure of the micro-jets of liquid may be controlled in order to ensure, for example, that the micro-jet reaches (and comes to a halt in) the dermis layer of the skin. This may be optimal for collagen remodelling, given that the dermis layer of the skin comprises substantial amounts of collagen and elastin. In some embodiments, a greater level of specificity may be optimal or required, e.g., it may be optimal that the micro-jet reaches the reticular dermis layer. For a given subject and/or procedure, there may exist an optimal depth for the liquid to reach. In some cases, the optimal depth may depend on characteristics of the subject, e.g., the subject's age, properties of the subject's skin, etc. and/or on characteristics of the procedure, e.g., what level of collagen remodelling is required or desired. Present methods of collagen remodelling using needleless injection of hot liquid, wherein the depth of the liquid injection may be controlled, may allow for substantially improved control over the energy deposit depth in the skin when compared to alternative methods.

The volume of liquid in the micro-jets of liquid may be controllable or selectable (e.g., by a controller). This may additionally or alternatively be described as the mass of the micro-jets. The volume may be increased to increase the strength of the injury or micro-burn that the injection step produces, since a greater volume may increase the total amount of energy transferred to the skin. This may, in some cases, increase production of collagen in the skin. It may additionally cause greater injury trauma. Conversely, the volume may be decreased to decrease the strength of the injury or micro-burn that the injection step produces. This may, in some cases, cause less injury trauma. It may additionally decrease production of collagen in the skin. For a given subject and/or procedure, there may exist an optimal volume for each micro-jet. In some cases, the optimal volume may depend on characteristics of the subject, e.g., the subject's age, properties of the subject's skin, etc. and/or on characteristics of the procedure, e.g., what level of collagen remodelling is required or desired. Present methods of collagen remodelling using needleless injection of hot liquid, wherein the micro-jet volume may be controlled, may allow for substantially improved control over the amount of energy deposited in the skin when compared to alternative methods.

In some embodiments, method 100 may further comprise activating, by a switch unit of the device for operation by a user, the needleless injection unit to allow injection of liquid; and deactivating, by the switch unit, the needleless injection unit to disallow injection of liquid. In some embodiments, the switch unit comprises a foot pedal. A foot-pedal may allow for hands-free operation of the needleless injection unit by a user.

The needleless injection unit may comprise a handheld needleless injection pen configured to be held by a user.

The liquid may comprise any suitable liquid. The liquid may, for example, be water or oil. The liquid may, for example, be a liquid solution, such as a water-based solution or an oil-based solution. The liquid may be a liquid that is non-toxic to the body of the subject. The liquid may be a liquid that the body of the subject is able to easily process or remove. In some embodiments, the liquid may instead be a gas or another substance with fluid properties.

The liquid may comprise a therapeutic serum and/or a serum for reducing the appearance of micro-burns and/or skin damage. In some embodiments, the serum may have a therapeutic or healing effect (and in other embodiments, it may not). However, the therapeutic effect may, in some embodiments, only be of substantial benefit when treating the micro-burns caused by contact with the hot serum. As such, injection of the serum may in some embodiments, for example those directed to cosmetics and/or improving the bodily appearance of a subject, have no substantial overall therapeutic, healing, or medical effect. Therefore, embodiments of the invention which involve using a serum as the liquid, for example those directed to cosmetics and/or improving the bodily appearance of a subject, may not constitute methods for treatment of the human or animal body by surgery or therapy.

In other embodiments, the serum may have an overall therapeutic effect, for example, if its therapeutic or healing effect is substantially greater than the damage of the micro-burns caused by the hot serum.

In some embodiments, the serum may only be for reducing the appearance of damage/micro-burns, e.g., for reducing redness of the skin.

The method as described above may be implemented using a suitable collagen remodelling device. A suitable collagen remodelling device may be required to comprise at least a needleless injection unit and a means for heating liquid which the needleless injection unit is configured to inject into the skin of the subject. FIGS. 2 and 3 describe an embodiment of a collagen remodelling device with a separate liquid reservoir (separated construction). FIGS. 4-6 describe an embodiment of collagen remodelling device with an integrated liquid reservoir (compact construction). Each embodiment is merely exemplary and is not intended to be limiting in terms of its exact implementation. Features may be taken from one embodiment and incorporated into another.

FIG. 2 schematically illustrates a repetitive needleless injection device, in accordance with an embodiment of the present invention. FIG. 3 schematically illustrates a controller of the repetitive needleless injection device shown in FIG. 2.

Repetitive needleless injection device 10 includes handheld unit 12. Although components of repetitive needleless injection device 10 are shown in the schematic drawing as outside of handheld unit 12, at least in some cases, those components may be enclosed within handheld unit 12.

Handheld unit 12 may include a casing that encloses components of repetitive needleless injection device 10 that are operable to repetitively eject a series of liquid micro-jets 30. Handheld unit 12 may have the general form of an elongated cylinder. For example, the shape of handheld unit 12 may be similar to that of a pen, syringe, pistol barrel, or similar handheld or manipulable object. When components of repetitive needleless injection device 10 are not enclosed within handheld unit 12, handheld unit 12 may be connected to those external components via a suitable flexible connection. The flexible connection may be configured to enable sufficiently free manipulation of handheld unit 12 so as not to impede injection of material contained in liquid micro-jets 30 into the skin in different applications.

Handheld unit 12 may enclose propulsion mechanism 13 and pressure cell 24. Propulsion mechanism 13 is configured to apply a series of pressure pulses to a liquid that fills pressure cell 24. As a result of application of each pressure pulse, a liquid micro-jet 30 may be ejected from pressure cell 24 via dispenser nozzle 26. The liquid may be of a temperature configured to cause burns/micro-burns to the skin of a subject when the liquid is in contact with the skin of the subject. For example, the liquid temperature may be between 65° C.-80° C. Each ejected liquid micro-jet may, therefore, be configured to cause a micro-burn when in contact with the skin. An ejected micro-jet which is incident on the skin may penetrate to a specific depth, which may depend on the pressure of the pressure pulse. For example, the micro-jet may penetrate to the epidermis, the dermis, or the hypodermis of the skin, as may be required.

Propulsion mechanism 13 may include impulse generator 14 and plunger 16. Impulse generator 14 may include an actuator that is operable to produce an impulse (e.g., move a surface such as actuation surface 18 in a linearly outward direction). Plunger 16 may be configured to be displaced linearly so as to transmit the impulse to pressure cell 24.

Plunger 16 may be configured to move linearly back and forth within a longitudinal dimension of handheld unit 12, as indicated by piston motion arrow 22. Plunger 16 may be configured to move in a distal direction (toward nozzle 26) in response to a displacement of actuation surface 18 of impulse generator 14. Impulse generator 14 may be configured to displace actuation surface 18 in response to a driver signal that is generated by actuator driver 56 of controller 40 of repetitive needleless injection device 10. Actuator driver 56 may generate driver signals with a repetition rate that is determined by operation of triggering oscillator 54 of controller 40.

Actuator driver 56 may control operation of impulse generator 14 via actuator connection 37. For example, actuator connection 37 may include an electric cable, e.g., a lightweight electric cable. In some cases, e.g., where handheld unit 12 includes a self-contained power supply, actuator connection 37 may include a wireless connection.

For example, impulse generator 14 may include a piezoelectric actuator, a magnetostrictive actuator, pulsed laser and a material that is configured to expand upon absorption of a laser pulse, an actuated high-pressure vessel, a linear electromagnetic motor, a compressed mechanical spring, or another type of actuator that may be driven at a suitable repetition rate. A preference or requirement for a particular repetition rate may be determined in accordance with an intended application of repetitive needleless injection device 10. For example, a repetition rate may be selected so as to enable delivery of a sufficient amount of a material (e.g., a serum and/or a liquid) to the skin at a desired rate (e.g., during a comfortable or natural rate of movement of handheld unit 12 over the skin surface, or an otherwise determined rate).

An impulse generator 14 in the form of a piezoelectric actuator may include a piezoelectric crystal connected to suitable electrodes. The maximum displacement of a surface of the piezoelectric crystal may not be sufficient to enable expulsion of a liquid micro-jet 30. In such a case, impulse generator 14 may include a mechanical amplifier. The mechanical amplifier may be configured to produce a sufficiently large displacement of actuation surface 18 in response to a smaller displacement of a surface of the piezoelectric crystal that is applied to the mechanical amplifier. For example, actuation surface 18 may represent a surface of the mechanical amplifier with amplified displacement, or a surface that is mechanically coupled to such a surface of the mechanical amplifier. Similarly, an impulse generator 14 that includes a magnetostrictive or other type of actuator may include a mechanical amplifier.

For example, a mechanical amplifier may include an elliptical cell, an arrangement of one or more levers, or another type of mechanical amplifier. For example, the amplification factor of the mechanical amplifier may be about 10 (e.g., for a piezoelectric actuator), or another suitable amplification factor.

Actuation surface 18 may be configured to apply a force to proximal end 16a to push plunger 16 in the distal direction. The force is transmitted to liquid in pressure cell 24 by distal end 16b of plunger 16. Thus, the force that is transmitted by plunger 16 may increase the pressure of the liquid in pressure cell 24 over the pressure that is applied by the ambient atmosphere.

Pressure cell 24 may be configured such that the only outlet of liquid from pressure cell 24 under application of excess pressure is orifice 27 of nozzle 26. For example, a diameter of distal end 16b of plunger 16 may be slightly less than the interior diameter of pressure cell 24. Any space between the perimeter of distal end 16b and the interior walls of pressure cell 24 may be filled with sealing structure (e.g., O-ring or other sealing structure). The sealing structure may include a low friction surface so as to prevent liquid flow between distal end 16b and walls of pressure cell 24 without unduly impeding motion of plunger 16.

Structure of pressure cell 24 or of plunger 16 may be configured to prevent backflow of liquid from pressure cell 24 to reservoir 32 during application of excess pressure to pressure cell 24. For example, an inlet conduit 34 for conducting the liquid from reservoir 32 to pressure cell 24 may include inlet unidirectional valve 36. Inlet unidirectional valve 36 may be configured to enable flow of fluid from reservoir 32 to pressure cell 24 when suction is applied to pressure cell 24, while preventing backflow of liquid from pressure cell 24 toward reservoir 32. For example, inlet unidirectional valve 36 may be located at an interface between inlet conduit 34 and pressure cell 24, as shown. Alternatively or in addition, inlet unidirectional valve 36 may be located at an interface between reservoir 32 and inlet conduit 34, or elsewhere along inlet conduit 34. Alternatively or in addition, e.g., when reservoir 32 is enclosed within handheld unit 12, one or more of reservoir 32, plunger 16, or pressure cell 24 may be configured to seal off flow between reservoir 32 and pressure cell 24 when pressure is applied to pressure cell 24.

Since the only outlet from pressure cell 24 is orifice 27 of nozzle 26, the excess pressure may force the liquid out of pressure cell 24 via orifice 27 in the form of a liquid micro-jet 30. The ejection of liquid micro-jet 30 may relieve the excess pressure in pressure cell 24, restoring an equilibrium state where the pressure of the liquid is countered by retaining forces (e.g., atmospheric pressure, adhesion, surface tension, or other forces at orifice 27).

After the distal displacement of actuation surface 18, impulse generator 14 may retract actuation surface 18 in the proximal direction (away from nozzle 26). The retraction displaces actuation surface 18 to substantially the original position of actuation surface 18 prior to the distal displacement. When actuation surface 18 is retracted, one or more restoration mechanisms similarly retract plunger 16 to its original proximal position.

For example, the restoration mechanism may include a rigid bond of plunger 16 to impulse generator 14, e.g., at actuation surface 18. The rigid bond may be formed as one piece with part of impulse generator 14 (e.g., by casting, moulding, or extruding plunger 16 and a part of actuation surface 18 or of impulse generator 14 as a single piece, or by machining a single piece to form them). The rigid bond may include a bonding material (e.g., adhesive, glue, cement, epoxy, solder, or other bonding material) a mechanical fastener (e.g., screw, clamp, or other mechanical fastener), magnetic attraction, or another rigid connection. Thus, the retraction of actuation surface 18 entails retraction of the connected plunger 18. Such a rigid bond may enable precise control of the position of the plunger by controlling operation of impulse generator 14. (Such precise control may be especially advantageous in a repetitive needleless injection device 10 that does not include an outflow unidirectional valve 28.)

Alternatively or in addition, the restoration mechanism may include retraction mechanism 20. Retraction mechanism 20 may include a resilient element such as a spring or deformable gasket, a magnet, or another element, that exerts a restoring force on plunger 16 in the proximal direction. Thus, after a pushing force of actuation surface 18 on proximal end 16a of plunger 16 is released, possibly separating actuation surface 18 from plunger 16, retraction mechanism 20 may push plunger 16 in the proximal direction.

When a retraction mechanism 20 is used without a rigid connection between actuation surface 18 and plunger 16, plunger 16 may separate from actuation surface 18 after exertion of a pushing force. For example, inertial of plunger 16 may cause proximal end 16a to separate from actuation surface 18. Such separation may result in the amplitude of the motion of plunger 16 being greater than that of the motion of actuation surface 18. The increased amplitude of the displacement may further increase the amount of liquid that is forced out of nozzle 26 by application of the pressure. The micro jet velocity can be increased by increasing the pressure in the pressure cell 24, for example, by increasing the power of the impulse generator 14 (e.g., increasing voltage on the piezoelectric actuator). The increased velocity of liquid micro-jet 30 may increase the depth of penetration of liquid micro-jet 30 into the skin. The increased volume of liquid micro-jet 30 may increase the resultant dose to the skin of a material that is delivered by liquid micro-jet 30.

Retraction of plunger 16 by the restoration mechanism after expulsion of liquid micro-jet 30 may create suction in pressure cell 24. The suction may draw liquid from reservoir 32 into pressure cell 24 via inlet conduit 34 and inlet unidirectional valve 36. Alternatively or in addition, when plunger 16 is retracted, liquid may flow from reservoir 32 into pressure cell 24 via one or more openings that are opened by retraction of plunger 16.

Inflow of air via orifice 27 of nozzle 26 during application of suction to pressure cell 24 may be prevented by outlet unidirectional valve 28 that is configured to control flow through nozzle 26. Outlet unidirectional valve 28 is configured to enable expulsion of a liquid micro-jet 30 from pressure cell 24 through orifice 27 when excess pressure is applied to pressure cell 24. Outlet unidirectional valve 28 is also configured to prevent inflow, e.g., of atmospheric air, through orifice 27 of nozzle 26 into pressure cell 24 when suction is applied to pressure cell 24. Thus, when suction is applied to pressure cell 24, inflow is enabled only from reservoir 32.

Alternatively or in addition to action of outlet unidirectional valve 28, inflow of air through orifice 27 of nozzle 26 and into pressure cell 24 during application of suction to pressure cell 24 may be prevented by adhesive forces and surface tension (or, collectively, capillary forces) that act on liquid in orifice 27. If the force of the applied suction on liquid in orifice 27 is less than the capillary forces, inflow of air through orifice 27 may be prevented. In this case, outlet unidirectional valve 28 may not be needed. For example, the capillary force may be expressed as Hγcosθ, where H is the circumference of the inner surface of orifice 27, γ is the surface tension of the liquid in orifice 27 (e.g., in units of force per length), and θ is the fluid contact angle of the liquid in orifice 27 with the interior walls of orifice 27 (dependent on adhesive forces between the liquid and the material of the interior wall of orifice 27).

The flow of liquid from reservoir 32 into pressure cell 24 may replace the volume of liquid that was ejected from pressure cell 24 in liquid micro-jet 30. Replenishing the liquid in pressure cell 24 may restore pressure cell 24 to an equilibrium state.

A cycle of operation of repetitive needleless injection device 10 includes operation of pushing plunger 16 to apply a pulse of excess pressure to pressure cell 24 to expel a liquid micro-jet 30, and retraction of plunger 16 to create a suction to replenish the supply of liquid in pressure cell 24. The time required to complete this cycle is the cycle time of repetitive needleless injection device 10. For example, the cycle time may be about 1 millisecond. In this case, the maximum repetition rate for a series of cycles is about 1000 hertz. A repetition rate of about 1000 Hz may be ideal to apply a serum and/or a liquid at a rate that is suitable for such applications as collagen remodelling. Other repetition rates may also be used.

Liquid reservoir 32 may include a liquid container vessel that is open to atmospheric pressure at opening 33. In this case, reservoir 32 may include a stationary container that is connected to pressure cell 24 by a flexible inlet conduit 34. For example, a flexible inlet conduit 34 may include a tube that is made of a flexible plastic or similar material. Alternatively or in addition, inlet conduit 34 may be constructed of a plurality of rigid tubes that are connected by flexible joints. The flexibility of inlet conduit 34 may enable free manipulation of handheld unit 12 while maintaining the fluid connection of pressure cell 24 to reservoir 32.

When reservoir 32 is open to atmospheric pressure and enclosed within handheld unit 12, opening 33 may be located on a side of handheld unit 12 that is designated to face upward. For example, handheld unit 12 may include a grip or other structure to facilitate maintaining an orientation of handheld unit 12 where opening 33 faces upward. Alternatively or in addition, opening 33 may be provided with baffles, unidirectional valves, or other structure to inhibit or prevent outward spillage of liquid from reservoir 32 via opening 33. In some cases, opening 33 may be covered by a flexible membrane that transmits pressure while preventing spillage.

In some cases, reservoir 32 may be provided with a liquid level sensor 38 to measure liquid level 31 of liquid in reservoir 32. For example, liquid level sensor 38 may be configured to generate a signal that is indicative of a sensed position (e.g., indicated by a sensed height, volume, pressure, electrical resistance, dielectric constant, radiation attenuation, refraction, heat conduction, or other quantity that may be indicative of liquid level 31) of liquid level 31. The generated signal may be transmitted via sensor connection 35 to controller 40. Sensor connection 35 may include an electric cable (e.g., a lightweight cable for transmitting a low voltage signal) or a wireless connection. Alternatively or in addition, a counter or counting mechanism or function may be provided to count the number of pulses that were applied by operation of impulse generator 14. If at least an approximate volume of each ejected micro-jet 30 is known, a volume of the liquid that remains in reservoir 32 may be estimated.

In some embodiments, reservoir 32 may be provided with a heater 48, e.g., an electric heater or filament, which may be configured to heat any liquid contained therein. The heater may be powered and/or controlled by a heater connection 47. The heater connection 47 may include an electric cable for carrying an electric current. The electric current may convey power and/or information.

The reservoir may also be provided with a thermometer 46 configured to detect the temperature of any liquid therein. The thermometer may be configured to generate a signal that is indicative of a temperature of the liquid. The generated signal may be transmitted via sensor connection 45 to controller 40. Sensor connection 45 may include an electric cable (e.g., a lightweight cable for transmitting a low voltage signal) or a wireless connection.

The heater may be configured to heat a liquid to a temperature configured to cause micro-burns to the skin of the subject when the liquid is in contact with the skin of the subject. For example, the heater may heat the liquid in the reservoir to a temperature of approximately 65° C.-80° C. Different temperatures may be selected. Temperatures may be selected using controller 40. The heater may be configured to heat the liquid in the reservoir until the thermometer indicates that an appropriate temperature has been reached. At this point, the heater may turn off until the thermometer indicates that the temperature of liquid in the reservoir has fallen below an unacceptable level.

Controller 40 (e.g., circuitry of controller 40 or a processor 52 of controller 40 operating in accordance with programmed instructions that are stored on data storage device 58) may be configured to stop operation of impulse generator 14 (e.g., by controlling operation of triggering oscillator 54 or of actuator driver 56) when liquid level 31 falls below a predetermined value. For example, the predetermined value may be a level that is sufficient to prevent air bubbles from forming in inlet conduit 34 or in pressure cell 24.

Alternatively or in addition, controller 40 may be configured to generate an alert when liquid level 31 falls below a predetermined threshold level. For example, the generated alert may be output (e.g., by producing a visible or audible indication using output device 44) to inform a user of repetitive needleless injection device 10 that liquid level 31 is low. The user may stop operation of impulse generator 14 (e.g., by operating one or more user controls 42), may replenish the supply of the liquid in reservoir 32, may replace reservoir 32, or may perform another action in response to the generated alert.

Components of controller 40 may be external to handheld unit 12. For example, controller 40 may be connected to handheld unit 12 by a flexible wire or cable, or via a wireless connection. Alternatively or in addition, components of controller 40 may be enclosed within or mounted to handheld unit 12.

Controller 40 may include power supply 50. For example, power supply 50 may include one or more batteries, photovoltaic cells, or another self-contained power source. Power supply 50 may include one or more transformers or power converters to convert an electrical power signal from an external power source, e.g., from an electrical mains, generator, photovoltaic array, or another external power source to a power signal that is suitable for operation of one or more components of repetitive needleless injection device 10. In the case that components of controller 40 communicate wirelessly with components of handheld unit 12, handheld unit 12 may be directly provided with a separate supply of electric power (or a component of power supply 50).

Controller 40 may include a processor 52. For example, processor 52 may include one or more processing units, e.g. of one or more computers, that are configured to operate in accordance with programmed instructions. Alternatively or in addition, processor 52 may include analogue or digital circuitry that is configured to perform one or more operations, e.g., in a fixed manner in accordance with one or more input parameter values that are selected by operation of user controls 42.

In some cases, a processor 52 in the form of a processing unit may communicate with data storage device 58. Data storage device 58 may include one or more fixed or removable, volatile or non-volatile memory or data storage units. Data storage device 58 may include a computer readable media. Data storage device 58 may be utilized to store programmed instructions for operation of processor 52, data or parameters for use by processor 52 during operation, or results of operation of processor 52.

Processor 52 may be configured to receive signals from one or more sensors 57. For example, sensors 57 may include liquid level sensor 38 and thermometer 46. Sensors 57 may include one or more sensors that measure one or more conditions that could affect operation of repetitive needleless injection device 10. For example, sensors 57 may be configured to measure one or more of a temperature (e.g., of the ambient atmosphere, of liquid in pressure cell 24, of liquid in the reservoir, of the skin, or other temperature), a barometric pressure, relative humidity, a light or colour sensor (e.g., to monitor delivery of a serum and/or a liquid to the skin), a flowmeter (e.g., in inlet conduit 3 or elsewhere), a sensor to measure a property of a liquid in pressure cell 24 or in reservoir 32 (e.g., electrical or thermal conductivity, density, viscosity, pressure, colour, or another property), or other relevant properties. Processor 52 may be configured to control operation of repetitive needleless injection device 10 in accordance with the sensed values. A processor 52 in the form of a processing unit may be configured to interpret signals that are received from sensors 57 to obtain a measured value, to store signals or measured values on data storage device 58, or to utilize the measured values in controlling operation of one or more components of repetitive needleless injection device 10.

Processor 52 may be configured to operate triggering oscillator 54. Triggering oscillator 54 may include one or more clock circuits or oscillator devices. A frequency of operation of triggering oscillator 54 may be adjustable, e.g., by operation of one or more user controls 42. Adjustment of an oscillation rate of triggering oscillator 54 may determine a repetition rate for operation of impulse generator 14 of repetitive needleless injection device 10.

User controls 42 may include one or more dials, pushbuttons, switches, levers, sliders, knobs, keys, touch screens, pointing devices, keyboards, keypads, microphones, or other devices that are operable by a user to control operation of controller 40 and of repetitive needleless injection device 10. For example, user controls 42 may be operated to adjust one or more parameters that determine a state of repetitive needleless injection device 10 (e.g., operate, standby, off, or another state), delivered dose, a penetration depth of a delivered liquid into the skin, a repetition rate, a threshold liquid level, a temperature of the liquid, or another parameter of operation of repetitive needleless injection device 10.

A current setting may be displayed or otherwise output, e.g., via output device 44. Output device 44 may include one or more display screens, display panels, indicator lamps, speakers, printers, bells, buzzers, vibrators, or another device capable of producing visible, audible, or tactile output.

Processor 52 may be configured to operate actuator driver 56. Operation of actuator drive 56 may cause propulsion system 13 to generate a series of impulses that are applied to pressure cell 24. An impulse may be characterized by a set of parameters that describe displacement of propulsion system 13 as a function of time. For example, the component may include one or more of actuation surface 18 and plunger 16 (e.g., both when rigidly connected to one another).

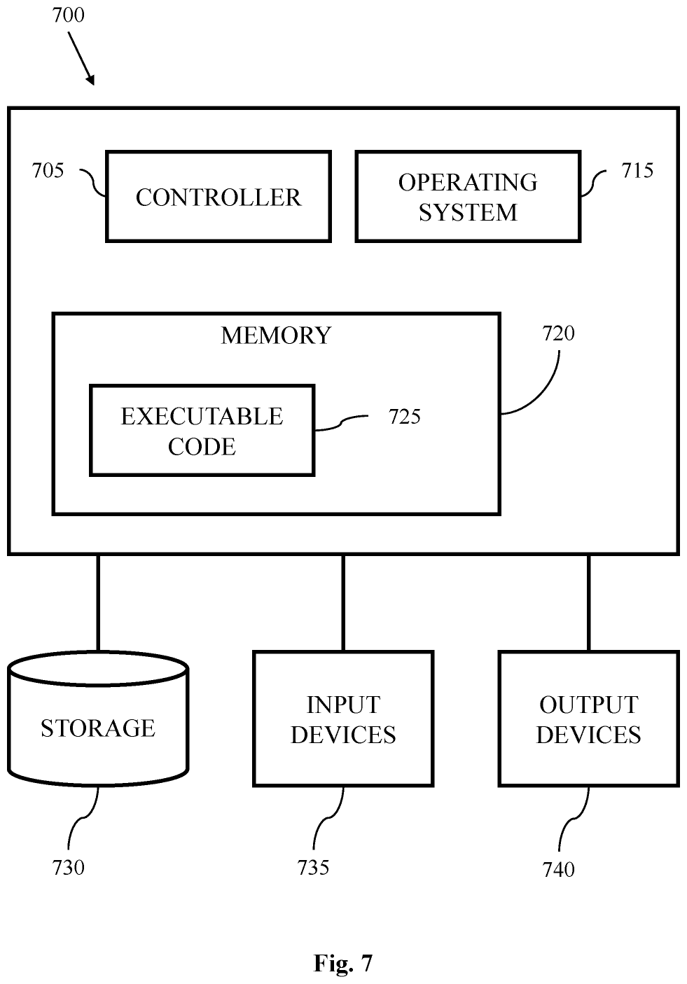

The computational components and/or computers of FIG. 3, e.g., controller 40, processor 50, data storage 58, sensors (input devices) 58, etc. may be as described with respect to FIG. 7.

FIG. 4 schematically illustrates a repetitive needleless injection device, in accordance with an embodiment of the present invention. In order to distinguish the repetitive needleless injection device of FIG. 4 from that of FIG. 2, the needleless injection device of FIG. 4 may be referred to as a compact repetitive needleless injection device. The needleless injection devices of FIGS. 2 and 4 are example embodiments, and features may be taken from one embodiment and incorporated into the other in accordance with specific requirements. While a controller is not shown in FIG. 4, a controller may be connected to or incorporated into the repetitive needleless injection device of FIG. 4 if required. Some of the components of FIG. 4 are shared with those in FIG. 2. These components may have the same reference numeral as in FIG. 2 and may be as described with respect to FIG. 2.

In compact needleless injection device 80, reservoir 32 is enclosed within handheld unit 12. For example, reservoir 32 may completely or partially fill a space between propulsion system 13 (e.g., between one or both of impulse generator 14 and plunger rod 82) and wall 78 of handheld unit 12. Opening 33 to reservoir 32 may enable refilling reservoir 32 and enables atmospheric pressure to propel liquid into pressure cell 24 when suction is applied to pressure cell 24. Opening 33 may be located on a side of handheld unit 12 that is designed to face upward. For example, handheld unit 12 may include a grip or other guiding structure to facilitate maintaining an orientation of handheld unit 12 where opening 33 faces upward. Alternatively or in addition, opening 33 may be provided with one or more of a cover, baffle, unidirectional valve, or other structure configured to impede or prevent outward spillage of liquid from reservoir 32 via opening 33.

Inlet conduit 34 for connecting reservoir 32 with pressure cell 24 may be internal to plunger rod 82. The distal end of plunger rod 82 may be configured to slide back and forth (distally and proximally) within distal neck 85 of handheld unit 12. The outer diameter of plunger rod 82 may be sufficiently close to the inner diameter of distal neck 85 so as to prevent or impede flow of liquid between plunger rod 82 and distal neck 85. Alternatively or in addition, sealing structure may be provided to prevent flow of liquid between plunger rod 82 and distal neck 85 while enabling plunger rod 82 to slide within distal neck 85.

The distal end of distal neck 85, beyond the distal end of inlet conduit 34, may form pressure cell 24.

Inlet conduit 34 may be provided with conduit opening 83. Conduit opening 83 is open to the interior of reservoir 32 to enable flow of liquid from reservoir 32 into inlet conduit 34. Air outlet opening 39 may be configured to enable escape of any trapped air from inlet conduit 34 to the ambient atmosphere so as to prevent formation of bubbles in inlet conduit 34. Inlet unidirectional valve 36 at the distal end of inlet conduit 34 may be configured to enable outflow of liquid from inlet conduit 34 to pressure cell 24. Inlet unidirectional valve 36 may further be configured to prevent liquid in pressure cell 24 from flowing into inlet conduit 34. Thus, when plunger rod 82 is pushed distally, excess pressure may be applied to liquid in pressure cell 24 so as to force a liquid micro-jet 30 to be expelled from orifice 27 of nozzle 26. When plunger rod 82 is retracted proximally, capillary forces may limit or prevent inflow of air into pressure cell 24 via orifice 27. Therefore, the retraction of plunger rod 82 may cause a suction that opens inlet unidirectional valve 36. When inlet unidirectional valve 36 is open, liquid may flow from reservoir 32 via inlet conduit 34 into pressure cell 24.

A diameter of orifice 27 may be selected in order to enable ejection of a liquid micro-jet 30 having a diameter in a predetermined range. For example, a wide diameter may enable coverage of a large area within a given period of time (e.g., when delivering a substance, such as a serum and/or a liquid, to a general region). On the other hand, a narrow diameter may enable tracing finer features on the skin (e.g., when delivering a substance such as a serum and/or a liquid to a narrow region of skin, such as a wrinkle). For example, the useful diameter of orifice 27 for this application may be no wider than 300 micrometres. The diameter of the orifice also affects ejection velocity of the micro jets.

For example, impulse generator 14 may include a piezoelectric actuator. The piezoelectric actuator may or may not include a mechanical amplifier. For example, in order to minimize the space occupied by impulse generator 14, a piezoelectric actuator may not include a piezoelectric crystal without a mechanical amplifier.

In compact needleless injection device 80 as shown, plunger rod 82 may be bonded to actuation surface 18 of impulse generator 14. When actuation surface 18 of impulse generator 14 is retracted in the proximal direction plunger rod 82 is also retracted.

The plunger rod 82 may be constructed from electrically conductive material(s). If the plunger rod is constructed from electrically conductive material, then eddy currents or hysteresis losses may form if the plunger rod is subject to an alternating magnetic field or flux. These eddy currents or hysteresis losses may act to heat the plunger rod, which may in turn, act to heat the liquid stored in the liquid reservoir 32. Therefore, in this embodiment, the liquid in the reservoir may heat up under an alternating magnetic field or flux.

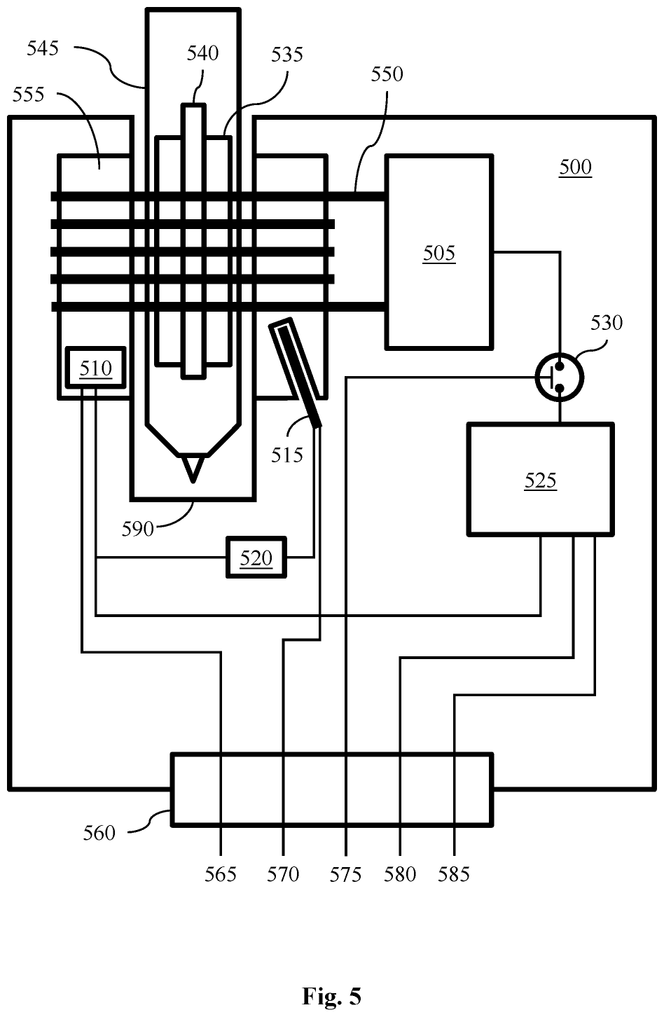

FIG. 5 shows a schematic diagram of a heater unit 500 and a needleless injection device/unit 545 of collagen remodelling device according to embodiments of the invention. FIG. 5 is an exemplary embodiment of a heater unit and needleless injection device; aspects of FIG. 5 may be removed or altered, as required. Heater unit 500 may heat liquid through a process of electromagnetic induction. The needleless injection unit may be as described in FIG. 4.

The heater unit may comprise an induction heater component 505, which may be configured to produce an alternating current (AC) through an electrically conductive coil 550. The heater unit may include an opening 590. The opening may be configured to house, hold, or contain the needleless injection unit during heating of a liquid.

The coil 550 may be wrapped or coiled around the opening 590. The opening may be surrounded by a block structure 555, which may be a toroid shape. The block structure may be a bobbin. The coil 550 may be wrapped around the block structure. When AC current is passed through the coil, an alternating magnetic field or flux may be generated within an area encompassed by the coil. Therefore, when AC current is passed through the coil, any electrically conductive or semi-conductive elements within the volume encompassed by the coil may experience eddy currents or hysteresis losses, which may act to heat any such electrically conductive or semi-conductive elements.

The needleless injection unit may comprise a liquid reservoir 535 for containing liquid, which may require heating. The needleless injection unit may also comprise an electrically conductive component 540, which may be in close proximity to or touching the liquid reservoir. The electrically conductive component may be a piston. The electrically conductive component may be plunger rod 82 of FIG. 4. When an alternating magnetic field or flux is generated within an area encompassed by the coil of the heater, the electrically conductive component may heat due to electrical currents being generated in the electrically conductive component. The electrically conductive component may then transfer heat (directly or indirectly) to the liquid contained in the liquid reservoir. The liquid may therefore be heated. In some embodiments, the extent of the coil may align with the liquid reservoir, e.g., the coil may have the same or similar height as the liquid reservoir. This may allow for improved uniformity of heating of the liquid.

In some embodiments, the block structure may additionally or alternatively be configured to heat up under the influence of an alternating magnetic field or flux. For example, the block structure may heat up if it comprises an electrically conductive material. In these embodiments, the block may then heat the liquid.

The heater unit may comprise a connector 560. The connector may allow for the heating unit to receive electrical signals and/or electrical power. Signals and/or power may be received from a controller. The connector may also allow for sending signals and/or power from the heater unit.

Electrical power may flow from a DC power connection 565 to a temperature switch 510, wherein the temperature switch may comprise a bimetal. The temperature switch may be embedded in the block structure. The temperature switch may be configured to deactivate if it reaches a high enough temperature. For example, the temperature switch may convey electricity until the temperature in its vicinity reaches the desired temperature of the liquid, at which point, the switch may deactivate and stop conveying electricity. Alternatively, the temperature switch may convey electricity until the temperature in its vicinity reaches a different temperature, for example a higher temperature than the desired temperature of the liquid. The temperature switch may be calibrated to deactivate at a temperature, which may indicate that the temperature of the liquid is at a desired level. Beyond the temperature switch, the DC power connection may continue (directly or indirectly) to any or all of: an induction heater, a relay, a DC-DC converter, an AC-DC converter, a voltage regulator, and a thermistor.

In addition to or as an alternative to the temperature switch, the heater unit may comprise a thermistor 515, or a resistor whose resistance is dependent on temperature. This thermistor is positioned in close proximity to the liquid reservoir 535 and made to maintain in thermal contact with the liquid reservoir. As such, its temperature, and therefore resistance, is strongly correlated with the liquid temperature. This allows a reliable estimation of the liquid temperature from the measured resistance of the thermistor, and, in turn, the control of the liquid's temperature by the controller 40. An electric current may be output from the heater unit from the thermistor, via an output connection 570. The resistance of the thermistor may be inferred from the output connection (e.g., by carrying out a calibration, or comparing the current of the output to an expected current or a current at the start of the heating process), and therefore, the temperature in the vicinity of the thermistor may be deduced (e.g., by the controller). The heater unit may, in some embodiments, be controlled accordingly. Power that is input to the thermistor may pass through a voltage regulator 520, for example a low dropout regulator (LDO). The voltage regulator may act to maintain a steady voltage passing through it. Power that is input into the thermistor and/or the voltage regulator may pass through the temperature switch or may arrive directly from the DC power connection.

In some embodiments, electric power may flow directly or indirectly (e.g., via temperature switch 510) to an optional DC-DC converter 525. A DC-DC converter may convert electric currents from one voltage level to another. A DC-DC converter may be adjustable, for example, the required output voltage may be adjustable. The DC-DC converter may be adjusted using an electric signal conveyed by a voltage control connection 585. The DC-DC converter may be provided with a ground connection (GND) 580. The voltage output by the DC-DC converter may be as required by the induction heater component (a higher voltage input into the induction heater may lead to more rapid heating of the liquid). The DC-DC converter may be advantageous for ensuring that a current that reaches the induction heater component has a steady voltage. The voltage/power output of the DC-DC converter may be directly or indirectly conveyed to the induction heater component. The voltage/power output of the DC-DC converter may be conveyed to the induction heater component via a relay switch 530, or any other suitable switch. The relay switch may provide a means for turning the heater on or off. The relay switch may be activated by an on/off signal received from an on/off connection 575 (which may be connected to a controller).

The induction heater component 505 may comprise an AC-DC converter configured to convert a received DC current into an AC current, which may be conveyed through the electrically conductive coil 550.

Many other embodiments of heater units are possible. Some heater units may be directly powered by AC currents. Some heater units may not have a temperature sensor or switch. In general, components of some heater units that operate via induction may include an opening, an electrically conductive coil encompassing the opening, and some means of conveying AC currents through the coil.

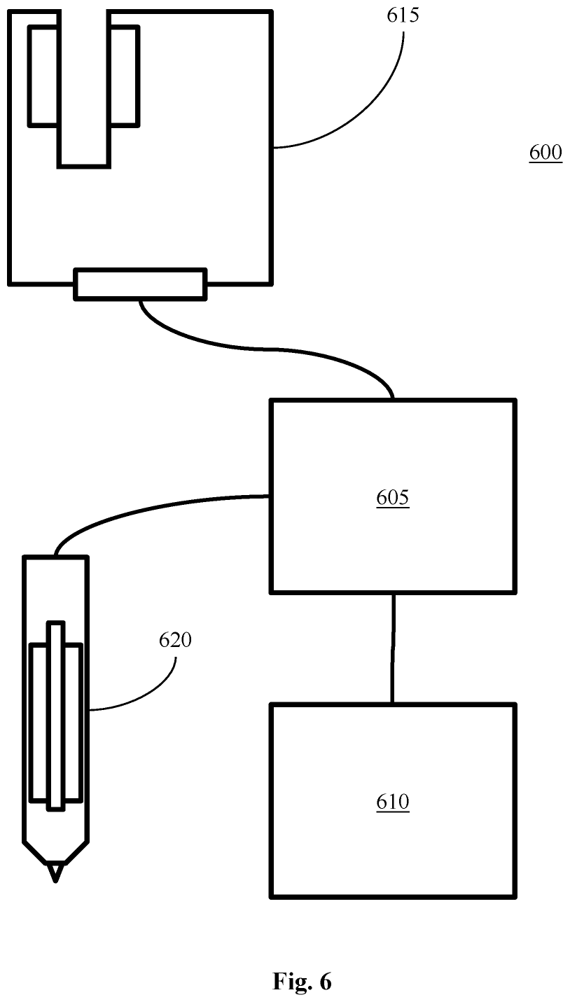

FIG. 6 shows a schematic diagram of a collagen remodelling device 600 according to some embodiments of the present invention.

The collagen remodelling device may comprise a controller 605. The controller may be the same or similar to the controller described with respect to FIG. 3 (and/or FIG. 7). The controller may control the function of a heater unit 615, which may be as described in FIG. 5, and may also control the function of a needleless injection unit 620, which may be as described in FIG. 4.

In some embodiments, the collagen remodelling device may further comprise a switch unit 610 for operation by a user. The switch unit may have an active state configured to cause the needleless injection unit to inject liquid, and an inactive state configured to cause the needleless injection unit to not inject liquid. The switch unit may convey electric signals to the controller indicating whether it is in an active or inactive state, and the controller may control the needleless injection unit accordingly. In some embodiments, the switch unit may comprise a foot pedal, for activation by the foot of a user. A foot pedal may allow for hands-free activation of the needleless injection unit by a user. In other embodiments, the switch unit may comprise any other suitable switch, such as a button. In some embodiments, the switch unit may be incorporated into or mounted on the needleless injection unit.

FIG. 7 shows a block diagram of an exemplary computing device which may be used with embodiments of the present invention. Computing device 700 may include a controller or computer processor 705 that may be, for example, a central processing unit processor (CPU), a chip or any suitable computing device, an operating system 715, a memory 720, a storage 730, input devices 735 and output devices 740 such as a computer display or monitor displaying for example a computer desktop system.

Operating system 715 may be or may include code to perform tasks involving coordination, scheduling, arbitration, or managing operation of computing device 700, for example, scheduling execution of programs. Memory 720 may be or may include, for example, a Random Access Memory (RAM), a read only memory (ROM), a Flash memory, a volatile or non-volatile memory, or other suitable memory units or storage units. At least a portion of Memory 720 may include data storage housed online on the cloud. Memory 720 may be or may include a plurality of different memory units. Memory 720 may store, for example, instructions (e.g., code 725) to carry out methods as disclosed herein, for example, methods for collagen remodelling. Memory 720 may use a datastore, such as a database.

Executable code 725 may be any application, program, process, task, or script. Executable code 725 may be executed by controller 705 possibly under control of operating system 715. For example, executable code 725 may be, or may execute, one or more applications performing methods as disclosed herein, such as monitoring interactions in real time. In some embodiments, more than one computing device 700 or components of device 700 may be used. One or more processor(s) 705 may be configured to carry out embodiments of the present invention by, for example, executing software or code.

Storage 730 may be or may include, for example, a hard disk drive, a floppy disk drive, a compact disk (CD) drive, a universal serial bus (USB) device or other suitable removable and/or fixed storage unit. Data described herein may be stored in a storage 730 and may be loaded from storage 730 into a memory 720 where it may be processed by controller 705. Storage 730 may include cloud storage.

Input devices 735 may be or may include a mouse, a keyboard, a touch screen or pad or any suitable input device or combination of devices. Output devices 740 may include one or more displays, speakers and/or any other suitable output devices or combination of output devices. Any applicable input/output (I/O) devices may be connected to computing device 700, for example, a wired or wireless network interface card (NIC), a modem, printer, a universal serial bus (USB) device or external hard drive may be included in input devices 735 and/or output devices 740.

Embodiments of the invention may include one or more article(s) (e.g., memory 720 or storage 730) such as a computer or processor non-transitory readable medium, or a computer or processor non-transitory storage medium, such as for example a memory, a disk drive, or a USB flash memory encoding, including, or storing instructions, e.g., computer-executable instructions, which, when executed by a processor or controller, carry out methods disclosed herein.

Systems and methods of the present invention may improve existing collagen remodelling technology. For example, the present invention may allow for accurately controlling the depth at which skin micro-burns are caused in order to target/damage only the areas of skin which require targeting in order to induce collagen remodelling. Controlling the depth at which micro-burns are caused may be achieved by changing the speed and/or pressure of ejected micro-jets. The present invention may also allow for accurately controlling the amount of energy deposited in order to cause damage or micro-burns, which may allow for controlling of the degree of damage caused. Controlling the amount of energy deposited may be achieved by altering the volume/mass of the micro-jets, and/or by changing the temperature of the micro-jets. Embodiments of the present invention may also allow for simultaneously causing damage or micro-burns to the skin and additionally treating the damage. Treating the damage may be cosmetic (e.g., reducing redness), medical (e.g., healing the burns), or both. Simultaneously causing damage and treating the damage may allow for causing collagen remodelling while additionally minimising cosmetic and/or medical damage to the skin. Simultaneously causing damage and treating the damage may be achieved by needleless injection of a serum (e.g., instead of a non-serum liquid).

Different embodiments are disclosed herein. Features of certain embodiments may be combined with features of other embodiments; thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for collagen remodeling of an area of skin of a subject using a collagen remodeling device, the method comprising:
heating, by a heater unit of the device, a liquid to a temperature configured to cause micro-burns to the skin of the subject when the liquid is in contact with the skin of the subject; and
injecting the liquid, using a needleless injection unit of the device, from a liquid reservoir of the device into the area of skin of the subject.

2. The method of claim 1, wherein the needleless injection unit comprises a handheld needleless injection pen configured to be held by a user.

3. The method of claim 1, wherein heating, by a heater unit of the device, a liquid to a temperature configured to cause micro-bums to the skin of the subject when the liquid is in contact with the skin of the subject comprises:
positioning the needleless injection unit in an opening of the heater unit;
passing an alternating current through at least one conductive coil, by a connected alternating current source, wherein the at least one conductive coil surrounds the opening, and thereby creating an alternating magnetic field in the opening;
heating, by the alternating magnetic field, a conductive or semi-conductive element of the needleless injection unit; and
transferring heat, from the conductive or semi-conductive element, to the liquid stored in the liquid reservoir, wherein the liquid reservoir is housed inside the needleless injection unit.

4. The method of claim 1, wherein the liquid comprises a therapeutic serum.

5. The method of claim 1 further comprising:
activating, by a switch unit of the device for operation by a user, the needleless injection unit to allow injection of liquid; and
deactivating, by the switch unit, the needleless injection unit to disallow injection of liquid.

6. The device of claim 5 wherein the switch unit comprises a foot pedal.

7. The method of claim 1, wherein the temperature configured to cause micro-burns to the skin of the subject is between 65° C.-80° C.

8. A collagen remodeling device for remodeling collagen in an area of skin of a subject, the device comprising:
a heater unit, for heating a liquid to a temperature configured to cause micro-burns to the skin of the subject when the liquid is in contact with the skin of the subject;
a liquid reservoir; and
a needleless injection unit, for injecting the liquid from the liquid reservoir into the area of skin of the subject.

9. The device of claim 8, wherein the needleless injection unit comprises a handheld needleless injection pen configured to be held by a user.

10. The device of claim 8, wherein:
the liquid reservoir is housed inside the needleless injection unit;
the needleless injection unit comprises a conductive or semi-conductive element configured to heat under an alternating magnetic field and transfer heat to the liquid stored in the liquid reservoir; and
the heater unit comprises:
an opening configured to house the needleless injection unit during heating of the liquid; and
at least one conductive coil surrounding the opening and connected to an alternating current source, wherein the at least one conductive coil is configured to create an alternating magnetic field in the opening when an alternating current passes through it.

11. The device of claim 8, wherein the liquid comprises a therapeutic serum.

12. The device of claim 8 further comprising a switch unit for operation by a user, wherein the switch unit has an active state configured to cause the needleless injection unit to inject liquid, and an inactive state configured to cause the needleless injection unit to not inject liquid.

13. The device of claim 12 wherein the switch unit comprises a foot pedal.

14. The device of claim 8, wherein the temperature configured to cause micro-burns to the skin of the subject is between 65° C.-80° C.

15. The device of claim 8, further comprising a control unit comprising a processor and a memory.

16. A method for improving the bodily appearance of an animal by remodeling collagen of an area of skin of the animal using a collagen remodeling device, the method comprising:
heating, by a heater unit of the device, a liquid to a temperature configured to cause micro-burns to the skin of the animal when the liquid is in contact with the skin of the animal; and
injecting the liquid, using a needleless injection unit of the device, from a liquid reservoir of the device into the area of skin of the animal.

17. The method of claim 16, wherein the animal is a human.

18. The method of claim 16, wherein the needleless injection unit comprises a handheld needleless injection pen configured to be held by a user.

19. The method of claim 16, wherein heating, by a heater unit of the device, a liquid to a temperature configured to cause micro-bums to the skin of the animal when the liquid is in contact with the skin of the animal comprises:
positioning the needleless injection unit in an opening of the heater unit;
passing an alternating current through at least one conductive coil, by a connected alternating current source, wherein the at least one conductive coil surrounds the opening, and thereby creating an alternating magnetic field in the opening;
heating, by the alternating magnetic field, a conductive or semi-conductive element of the needleless injection unit; and
transferring heat, by the conductive element, to the liquid stored in the liquid reservoir, wherein the liquid reservoir is housed inside the needleless injection unit.

20. The method of claim 16, wherein the liquid comprises a serum for reducing the appearance of micro-burns.

21-23. (canceled)
